(19) United States
(12) Patent Application Publication
(10) Pub. No.: US 2008/0027412 A1
(43) Pub. Date: Jan. 31, 2008

(54) Apparatus and methods for uterine anesthesia

(75) Inventors: Gerald Feuer, Atlanta, GA (US); Gerald J. Sanders, Sonoma, CA (US)

Correspondence Address: LUCE, FORWARD, HAMILTON & SCRIPPS LLP, 11988 EL CAMINO REAL, SUITE 200, SAN DIEGO, CA, 92130, US

(73) Assignee: FEMSPEC LLC, San Francisco, CA (US)

(21) Appl. No.: 11/493,103

(22) Filed: Jul. 25, 2006

(51) Int. Cl. A61M 31/00

(52) U.S. Cl. 604/515

(57) ABSTRACT

The present invention is related to an apparatus for uterine anesthesia that comprises an outer tube and an inner tube positioned within the outer tube. The apparatus is positioned in the cervical os of a patient and an anesthetic is dispensed through the inner tube. A stopper surrounding the inner tube prevents a drainage of the anesthetic through the interstice between the two tubes, while an applicator surrounding the outer tube prevents a loss of the anesthetic along the cervical walls and into the vaginal canal. The applicator also maintains the cervical os in a dilated position, operating as a tenaculum and providing access to the uterus by a clinician. Methods for providing uterine anesthesia are also provided.

### FIELD OF THE INVENTION

The present application relates to apparatus and methods for providing uterine anesthesia to a patient. More particularly, the present invention relates to apparatus and methods for injecting an anesthetic into to the uterus of a patient and for preventing the drainage of the anesthetic out of the uterus after injection.

### BACKGROUND OF THE INVENTION

Gynecological procedures that require access to the uterus are typically performed with the aid of an anesthetic, to minimize the discomfort to the patient during medical interventions that may be not only physically painful, but also psychologically traumatic.

These types of procedures begin with a dilation of the cervix. An antiseptic such as Betadine is first spread on the cervix, and a local anesthetic, such as 1-2% lidocaine or 1% chloroprocaine hydrochloride, may be administered in a paracervical fashion.

The cervix is then grasped with a single-toothed tenaculum to begin the dilation process, and is increasingly dilated with the aid of dilators, a process that may last several hours. Eventually a tube is inserted into the cervix to provide access to the uterus by a clinician.

Because of the extreme sensitivity of the uterine region, an anesthetic is injected into the uterus before the clinician performs any desired procedures. To deliver the anesthetic, a thin tube, commonly referred to as a pipelle in the medical profession, is first placed inside the tube already inserted into the cervix. The pipelle has one or more openings at the distal end for delivering the anesthetic, and is connected at the proximal end to an injection device, such as a syringe.

A common problem in this type of procedure is the drainage of the anesthetic out of the uterus after injection, which reduces the anesthetic available for numbing the uterine region. After the anesthetic is delivered, a counter-flow may occur within the interstice between the pipelle and the tube positioned in the cervical os, eventually dripping out of the proximal end of the tube. More anesthetic may be lost due to an imperfect contact between the tube positioned in the cervical os and the cervical walls, allowing the anesthetic to flow along the cervical walls and around the outer tube into the vaginal canal.

Apparatus have been disclosed in the prior art to prevent a drainage of the anesthetic out of the uterine region. U.S. Pat. No. 3,385,300 to Holter discloses a cervical cannula connected to a threaded tapered cone that is inserted in the cervical os. Holter's apparatus, does not extend beyond the cervical os and provides no means for injecting anesthetic directly into the uterus.

U.S. Pat. No. 4,430,076 to Harris discloses a combined uterine injector and manipulative device having an inflatable member at the insertable end, which seals the lower portion of the uterus to retain anesthetic injected into the uterine cavity. Harris's invention, however, provides no access to the uterus by a clinician after the inflatable member is engaged with the cervix.

U.S. patent application Ser. No. 6,423,038 to Vancaille discloses a topical intra-uterine anesthesia device having a curved catheter connected to an injection device. While the curved catheter decreases the risk of perforating the uterine walls, Vancaille's invention does not prevent the drainage of anesthetic out the uterus.

Therefore, it would be desirable to have apparatus and methods for delivering anesthetic to the uterine region that prevent the drainage of the anesthetic within the interstice between the pipelle and the outer tube.

It would also be desirable to have apparatus and methods for delivering anesthetic to the uterine region that prevent the loss of the anesthetic along the cervical walls and into the vaginal canal.

It would further be desirable to have apparatus and methods for delivering anesthetic to the uterine region that avoid the use of spiked tenacula, making the gynecologic procedure less traumatic for the patient.

### SUMMARY OF THE INVENTION

In view of the foregoing, it is an object of the present invention to provide apparatus and methods for uterine anesthesia that enable the dispensation of anesthetic to the uterine region without a significant loss of the anesthetic.

It is another object of the present invention to provide apparatus and methods for uterine anesthesia that prevent the drainage of the anesthetic into the interstice between the delivery pipelle and the outer tube surrounding the pipelle.

It is also an object of the present invention to provide apparatus and methods for uterine anesthesia that prevent the loss of the anesthetic along the cervical walls and the outer tube surrounding the pipelle.

It is a further object of the present invention to provide apparatus and methods for gynecological procedures that enable for the delivery of an anesthetic medication, provide access to the uterus by a clinician, and at the same time eliminate the use of spiked tenacula.

These and other objects of the present invention are accomplished by providing an apparatus for uterine anesthesia that comprises an outer tube and an inner tube positioned within the outer tube. A stopper surrounding the inner tube prevents the drainage of the anesthetic into the interstice between the two tubes, and an applicator surrounding the outer tube and positioned in the cervical os prevents the loss of the anesthetic along the cervical walls and into the vaginal canal. The applicator also maintains the cervical os in a dilated position, operating as a tenaculum.

In a preferred embodiment, the apparatus for uterine anesthesia constructed according to the principles of the present invention includes an outer member and an inner member reciprocating inside the outer member. The outer member includes an outer tube having a lumen that extends between the distal and proximal ends of the outer tube, and also includes an applicator disposed around the distal end of the outer tube. The applicator is shaped to fit into the cervical os of a patient and operates like a plug against the cervical walls, preventing flows of fluid between the uterine cavity and the vaginal canal.

The inner member includes an inner tube having a lumen extending between the distal and the proximal ends of the inner tube, and also includes a stopper disposed around the inner tube and dimensioned to allow the inner member to reciprocate within lumen of the outer tube while preventing fluid communication within the interstice between the inner and outer tubes.

In a preferred embodiment, the lumen of the outer tube tapers inwardly from the proximal end to the distal end to increase the sealing action of the stopper, as the inner tube translates in the direction of the uterus.

The applicator in the outer member preferably has a frustoconical shape and is made of a silicone material. One or more ridges may be disposed on the outer surface of the applicator to improve anchoring to the walls of the cervical os.

The stopper may also have a frustoconical shape, but in different embodiments, the stopper may be ring-shaped or have still other shapes. The stopper may also be made of a silicone material, or may be manufactured from an elastomeric material, a foam material, or any other suitable materials.

In different embodiments, the distal end of the inner tube may be open to enable the ejection of the anesthetic, or may be closed and rounded, to minimize the risk of puncturing the uterus due to an excessively forceful contact. In this case, the anesthetic would be ejected through one or apertures communicating with the lumen of the inner tube and disposed in the proximity of the distal end of the inner tube.

Methods for dispensing uterine anesthesia are also provided.

### BRIEF DESCRIPTION OF THE DRAWINGS

The above and other objects of the present invention will be apparent upon consideration of the following detailed description, taken in conjunction with the accompanying drawings, in which:

FIG. 1 is a cross-sectional view of a first embodiment of the present invention;

FIG. 2 is a front view of a second embodiment of the present invention, with a detailed view of the stopper connected to in the inner tube; and

FIGS. 3A and 3B are front views of different variants of the stopper attached to the inner tube.

### DETAILED DESCRIPTION OF THE INVENTION

The present invention is related to apparatus and methods for delivering an anesthetic medication to the uterine region and for preventing the undesired drainage of the anesthetic out of the uterine region. In a preferred embodiment, the anesthetic is injected through a pipelle that translates within the lumen of an outer tube and that is surrounded by a stopper creating a seal with the lumen of the outer tube. The anesthetic is then prevented from draining through the interstice between the pipelle and the lumen. An applicator maintains the outer tube anchored within the cervical os and prevents a loss of the anesthetic along the cervical walls.

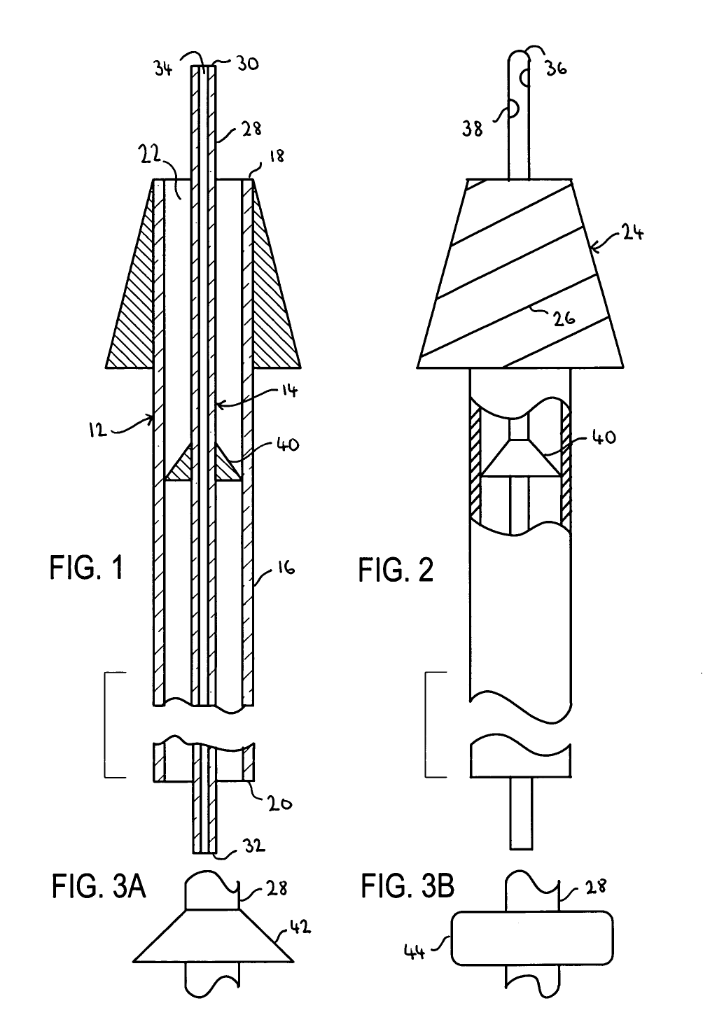

Referring to FIG. 1, an exemplary embodiment of an apparatus for uterine anesthesia constructed in accordance with the principles of the present invention is described. Apparatus for uterine anesthesia 10 includes two basic components, outer member 12 and inner member 14. In turn, outer member 12 includes outer tube 16 having distal end 18, proximal end 20 and lumen 22 that extends between distal end 18 and proximal end 20; and applicator 24 that is disposed around outer tube 16, typically in the proximity of distal end 18.

Outer tube 16 is of a length and rigidity sufficient to enable insertion of distal end 18 into the vaginal canal until contact with the cervical os is made. Outer tube 16 typically has an inner diameter between 3 and 9 mm, preferably 7 mm, and is typically made of a biocompatible material, such as surgical-grade polypropylene. A fitting, such as a Luer fitting, may be attached to proximal end 20 to enable connection to different types of medical equipment, for example, a syringe if manual aspiration is required, or a vacuum aspirator, if a mechanical aspiration is required.

Applicator 24 is disposed around outer tube 12, in the proximity of distal end 18, and is preferably frustoconical in shape. Applicator 24 is preferably made of a resilient material, for example, a silicone material, that maintains its basic shape during insertion and during the entire gynecological procedure, but also that is not as harsh on the patient's body as a metal or glass applicator. Additionally, a silicone applicator can be produced industrially at low cost, for example, by an injection molding process, making it economically feasible to dispose of it after use.

Referring now to FIG. 2, applicator 24 may have an outer surface that is smooth, or that comprises a plurality of ridges 26 that anchor applicator 24 to the walls of the cervical os, making applicator 24 act as a tenaculum, but without the perforations, bleeding and patient discomfort associated with a spiked tenaculum. Ridges 26 may be shaped in a variety of contours and may be disposed on the outer wall of applicator 24 in a variety of patterns. Preferably, ridges 26 are contoured as grooves and are disposed on the outer wall of applicator 24 in a spiral pattern, providing applicator 24 with screw-like shape that naturally adheres to the inner cervical walls. One skilled in the art will appreciate that other contours and lay-out patterns of ridges 26 are possible and still within the scope of the present invention, for example, ridges 26 may be contoured as rounded protrusions and be disposed in a circular parallel pattern.

Inner member 14 includes inner tube 28 having distal end 30, proximal end 32, and lumen 34 extending between distal end 30 and proximal end 32, and also includes stopper 40 disposed around inner tube 28 and creating a sealing contact between inner tube 28 and lumen 22. Inner tube 28 is employed for dispensing the anesthetic into the uterus and may be referred to as a “pipelle” within the medical industry. In the preferred embodiment, inner tube 28 has a diameter of 1-2 mm and is made of a biocompatible material, such as a surgical grade polypropylene.

A fitting may be attached to proximal end 32, providing for a suitable connection to a syringe or another type of injection device for dispensing the anesthetic. On the contrary, distal end 30 may be open, as shown in FIG. 1, to provide for an unimpeded outflow of the anesthetic, but is preferably closed with rounded tip 36, as shown in FIG. 2, to minimize the risk of puncturing the uterine walls in the event of an excessively forceful contact between distal end 30 and a uterine wall. When rounded tip 36 is employed, the anesthetic is released into the uterus through one or more apertures 38 connected to lumen 34 and disposed in the proximity of tip 36.

The numbing action of the anesthetic is proportional to the area of the uterus reached by the anesthetic. Accordingly, a drainage of the anesthetic away from the uterus during the injection process reduces the amount of anesthetic available inside the uterus, thus the effectiveness of the anesthetic.

One possible source of drainage is the interstice between inner tube 28 and the wall of lumen 22. To prevent such a drainage, stopper 40 is disposed around inner tube 28, creating a sealing contact with lumen 22. If the anesthetic drains into the interstice, stopper 40 provides a barrier that blocks the flow of the anesthetic, thereby causing the anesthetic to remain in the uterus.

Another source of drainage is the loss of the anesthetic between applicator 24 and the cervical walls, due to an imperfect contact between applicator 24 and the cervical walls. Because applicator 24 is inserted in the cervical os like a plug, any counter-flows along the cervical walls is prevented. The plugging action of applicator 24 is even more pronounced when ridges 26 provide a secure anchoring of applicator 24 to the cervical walls.

Stopper 40 may be configured in a variety of shapes. In one embodiment, shown in FIG. 3A, stopper 40 may have frustoconical shape 42 with an inner lumen, within which inner tube 28 is positioned. In another embodiment, shown in FIG. 3B, stopper 40 may be shaped like ring 44 surrounding outer tube 28. Stopper 40 may be manufactured from a variety of materials, such as silicone, foam, or an elastomeric material like rubber. However, one skilled in the art will appreciate that stopper 40 may be manufactured with a variety of other shapes and from a variety of other materials, all within the scope of the present invention.

In order to increase the effectiveness of stopper 40, lumen 20 may be tapered inwardly, with proximal end 20 narrower than distal end 18. For example, if lumen 22 has a 7 mm diameter at proximal end 20, lumen 22 may be tapered to a diameter measuring 6.8-6.9 mm at distal end 18. When lumen 22 is a tapered inwardly, inner tube 28 and stopper 40 advance within lumen 22 until the contact between stopper 40 and lumen 22 becomes increasingly tighter, requiring greater pressure by the clinician on proximal end 32 to advance inner tube 28. At the same time, the tighter contact between stopper 40 and lumen 22 provides for an increasingly effective sealing action.

A method for providing uterine anesthesia by the use of apparatus 10 is now described. As an initial step, apparatus 10 is assembled and prepared for use by placing inner member 14 within lumen 22 of outer member 14.

Outer member 22 is then inserted into the patient's vaginal canal, until applicator 24 contacts and becomes lodged in the patient's cervical os. To achieve the most appropriate positioning of applicator 24 within the walls of the os, the clinician may twirl applicator 24 until a proper anchoring is achieved. Ridges 26, if present, increase the anchoring action by penetrating into the walls of the os.

In the following step, inner tube 28 is advanced into the uterus, while applicator 24 maintains outer member 12 securely connected to the os. When distal end 34 or 36 reaches a position that the clinician deems most appropriate, anesthetic is injected through inner lumen 34, for example, by applying pressure on a syringe attached to proximal end 32. The anesthetic then exists lumen 34 and is released into the uterus.

The drainage of the anesthetic into the interstice between the wall of lumen 22 and inner tube 28 is also prevented by stopper 40, which blocks the passage of the anesthetic into the interstice. Additionally, the sealing effect provided by applicator 24 against the walls of the cervical os avoids a loss of the anesthetic along the walls of the os and into the vaginal cavity. Therefore, the anesthetic is constrained into the uterus, providing the most effective numbing action.

After an adequate amount of time has elapsed to allow the anesthetic to act, the inner tube is removed from the outer tube and may be discarded. The clinician is then able to implement any subsequent gynecological procedures by using the access provided by lumen 22.

While preferred embodiments of the invention are described above, it will be apparent to one skilled in the art that various changes and modifications may be made. The appended claims are intended to cover all such changes and modifications that fall within the true spirit and scope of the invention.

What is claimed is:

1. An apparatus for uterine anesthesia comprising:
an outer member including a first tubular portion having a first distal end, a first proximal end, and a first lumen extending therebetween, the outer member further including an applicator disposed around the first distal end, the applicator being shaped to fit into the cervical os of a patient and to prevent fluid communication between the uterine cavity and the vaginal canal through the cervical os; and
an inner member including a second tubular portion having a second distal end, a second proximal end, and a second lumen extending therebetween, the inner member further including a stopper disposed around the second tubular port-ion,
wherein the stopper is dimensioned to allow the inner member to reciprocate within the first lumen and to extend beyond the distal end of the outer member while preventing fluid communication between the first distal end and the first proximal end.

2. The apparatus of claim 1, wherein the first lumen has a diameter comprised between 3 and 9 mm.

3. The apparatus device of claim 1, wherein the first lumen tapers inwardly from the first proximal end to the first distal end.

4. The apparatus device of claim 1, wherein the applicator is of frustoconical shape.

5. The apparatus of claim 4, wherein the applicator is made of a silicone material.

6. The apparatus of claim 4, wherein the applicator includes one or more ridges disposed on its outer surface to improve anchoring of the applicator to the walls of the cervical os.

7. The apparatus of claim 6, wherein the ridges are defined by grooves carved on the applicator's outer surface and are disposed on the applicator's outer surface in a spiral pattern.

8. The apparatus of claim 1, wherein the second distal end is closed and rounded, and wherein one or apertures communicating with the second lumen are disposed in the proximity of the second distal end.

9. The apparatus of claim 1, wherein the stopper is frustoconical in shape.

10. The apparatus of claim 1, wherein the stopper is ring-shaped.

11. The apparatus of claim 1, wherein the stopper is made of a silicone, elastomeric, or foam material.

12. A method for uterine anesthesia comprising the steps of:
providing an outer member including a first tubular portion having a first distal end, a first proximal end, and a first lumen extending therebetween, the outer member further including an applicator disposed around the first distal end, the applicator being shaped to fit into the cervical os of a patient and to prevent fluid communication between the uterine cavity and the vaginal canal through the cervical os;
inserting an inner member into the first lumen, the inner member including a second tubular portion having a second distal end, a second proximal end, and a second lumen extending therebetween, the inner member further including a stopper disposed around the second tubular portion, wherein the stopper is dimensioned to allow the inner member to reciprocate within the first lumen and the extend beyond the distal end of the outer member while preventing fluid communication between the first distal end and first proximal end;
inserting the outer member into the vaginal canal and positioning the applicator in the cervical os;
advancing the second distal end towards the uterus to a position from which an anesthetic will be injected;
injecting the anesthetic in the uterus, the seal portion preventing a drainage of the anesthetic out of the first tubular portion; and
removing the inner member from the outer tube by reciprocating the second tubular member in the direction opposite to the uterus.

13. The method of claim 12, wherein the first lumen has a diameter comprised between 3 and 9 mm.

14. The method of claim 12, wherein the first lumen tapers inwardly from the first proximal end to the first distal end.

15. The method of claim 12, wherein the applicator is of frustoconical shape.

16. The method of claim 15, wherein the applicator is made of a silicone material.

17. The method of claim 15, wherein the applicator includes one or more ridges disposed on its outer surface to improve anchoring to the walls of the cervical os.

18. The method of claim 17, wherein the one or more ridges are shaped like grooves carved on the applicator's outer surface and are disposed on the applicator's outer surface in a spiral pattern.

19. The method of claim 12, wherein the second distal end is closed and rounded, and wherein the anesthetic is injected in the uterus through one or apertures communicating with the second lumen and disposed in the proximity of the second distal end.

20. The method of claim 12, wherein the stopper is frustoconical in shape.

21. The method of claim 12, wherein the stopper is ring-shaped.

22. The method of claim 12, wherein the stopper is made of a silicone, elastomeric, or foam material.
